SEQUENCE LISTING (18 sequences)

SEQ ID NO: 1 - DNA, 1008 bp, Colocasia esculenta cv. Kaosiung no. 1
cgcccgggcaggtcgcagcatcagtcgcggtgcttctgctgttgctgccaccgacctcgtcggagttcgtctcgggcgaagctttgaaggtggagtggggaggtggagacgatccagtgatcaggatggccttgatggggggcatcgtggatgtggagggagcgcagaacagcgccgaagtagaggagctcgcccgcttcgccgtcgacgaacacaataagaaggagaatgcgcttctgcagttctcccggcttgtgaaggcgaagcaacaggtggtttcgggcattatgcatcacctcactgtggaggttatcgaaggtggcaagaagaaagtgtatgaagccaaggtctgggtccaggcttggcttaactccaagaagcttcatgagttcagccccattggcgattcatcctcggttacgccagcagatctcggtgtaaaacgggatgcgcacgaagcggaatggctagagataccaacacatgatcctgtcgtccaagacgcagcaaaccatgcggtgaagagcatccagcaaagatccaatactttgttcccttatgaactgttggagatccttcatgcaaaggctaaggtgcttgaggacctcgccaaaatccatttgctactcaaacttaagaggggaagcagggaggagaagttcaaggtggaagtgcacaagaacattgaggggactttccatctgaatcagatggagcaagatcattcagactctggaaactaggatcgacggccgggtaagtgcttcgtgcacctgtaaaaaaaataattcgagtgtctgtattcccttatcatcgtactagcgtgtgtgaaaatgacaaaaacagtgctgtggagctttgcttatgatcctttattcgcctcgtactctttgactatgtctgcatcttttcctcctgcgatatttgcatgtatctcttcccgacattaagatttctatatacgaatatttggtaatcggccaaaaaaaaaaaaaaaaaaaaaaaaaa

SEQ ID NO: 2 - protein, 205 aa, Colocasia esculenta cv. Kaosiung no. 1
MALMGGIVDVEGAQNSAEVEELARFAVDEHNKKENALLQFSRLVKAKQQVVSGIMHHLTVEVIEGGKKKVYEAKVWVQAWLNSKKLHEFSPIGDSSSVTPADLGVKRDAHEAEWLEIPTHDPVVQDAANHAVKSIQQRSNTLFPYELLEILHAKAKVLEDLAKIHLLLKLKRGSREEKFKVEVHKNIEGTFHLNQMEQDHSDSGN

SEQ ID NO: 3 - DNA, 19 bp, Artificial Sequence
  Description of Artificial Sequence  Synthetic oligonucleotide primer
rrsctcgcbcgmttcgccg

SEQ ID NO: 4 - DNA, 18 bp, Artificial Sequence
  Description of Artificial Sequence  Synthetic oligonucleotide primer
cgcgtcgaygarcacaac

SEQ ID NO: 5 - protein, 10 aa, Colocasia esculenta cv. Kaosiung no. 1
LARFAVDEHN

SEQ ID NO: 6 - protein, 5 aa, Colocasia esculenta cv. Kaosiung no. 1
QVVSG

SEQ ID NO: 7 - DNA, 25 bp, Artificial Sequence
  Description of Artificial Sequence  Synthetic oligonucleotide primer
ttgatccatgcttgatggggggcat

SEQ ID NO: 8 - DNA, 28 bp, Artificial Sequence
  Description of Artificial Sequence  Synthetic oligonucleotide primer
ttgaatcctttccagagtctgaatgatc

SEQ ID NO: 9 - protein, 245 aa, Soybean
MRALTSSSSTFIPKRYSFFFFLSILFALRSSSGGCSEYHHHHAPMATIGGLRDSQGSQNSVQTEALARFAVDEHNKKQNSLLEFSRVVRTQEQVVAGTLHHLTLEAIEAGEKKLYEAKVWVKPWLNFKELQEFKPAGDVPSFTSADLGVKKDGHQPGWQSVPTHDPQVQDAANHAIKTIQQRSNSLVPYELHEVADAKAEVIDDFAKFNLLLKVKRGQKEEKFKVEVHKNNQGGFHLNQMEQDHS

SEQ ID NO: 10 - protein, 201 aa, Arabidopsis
MALVGGVGDVPANQNSGEVESLARFAVDEHNKKENALLEFARVVKAKEQVVAGTLHHLTLEILEAGQKKLYEAKVWVKPWLNFKELQEFKPASDAPAITSSDLGCKQGEHESGWREVPGDDPEVKHAAEQAVKTIQQRSNSLFPYELLEVVHAKAEVTGEAAKYNMLLKLKRGEKEEKFKVEVHKNHEGALHLNHAEQHHD

SEQ ID NO: 11 - protein, 207 aa, Brassica-oleracea
MAMLGGVRDLPANENSVEVESLARFAVDEHNKKENALLEFARVVKAKEQVVAGTMHHLTLEIIEAGKKKLYEAKVWVKPWLNFKELQEFKPASDDGAPSTTITPSDLGCKKVSDENASGWREVPGDDPEVQHVADHAVKTIQQRSNSLFPYELQEVVHANAEVTGEAAKYNMVLKLKRGEKEEKFKVEVHKNHEGVLHLNHMEQHHD

SEQ ID NO: 12 - protein, 209 aa, Castor-bean
MATVQGGVHDSPQGTANNAEIDGIARFAVDEHNKKENAMVEFGRVLKAKEQVVAGTLHHLTIEAIEAGKKKIYEAKVWVKPWLNFKELQEFKHATDVADTTASHPSFTSSDLGVKREGHGAEWKEVAAHDPVVQDAATHAVNTIQQRSNSLFPYQLQEIVHAKAQVVDDFAKFDMILKVKRGTSEEKFKVEVHKNNEGTFLLNQMEPHT

SEQ ID NO: 13 - protein, 102 aa, Oryzacystatin I
MSSDGGPVLGGVEPVGNENDLHLVDLARFAVTEHNKKANSLLEFEKLVSVKQQVVAGTLYYFTIEVKEGDAKKLYEAKVWEKPWMDFKELQEFKPVDASANA

SEQ ID NO: 14 - protein, 107 aa, Oryzacystatin II
MAEEAQSHAREGGRHPRQPAGRENDLTTVELARFAVAEHNSKANAMLELERVVKVRQQVVGGFMHYLTVEVKEPGGANKLYEAKVWERAWENFKQLQDFKPLDDATA

SEQ ID NO: 15 - protein, 81 aa, Maize
QLQELARFAVNEHNQKANALLGFEKLVKAKTQVVAGTMYYLTIEVKDGEVKKLYEAKVWEKPWENFKQLQEFKPVEEGASA

SEQ ID NO: 16 - protein, 134 aa, Maize
MRKHRIVSLVAALLILLALAVSSNRNAQEDSMADNTGTLVGGIQDVPENENDLHLQELARFAVDEHNKKANALLGFEKLVKAKTQVVAGTMYYLTIEVKDGEVKKLYEAKVWEKPWEKFKELQEFKPVEEGASA

SEQ ID NO: 17 - protein, 90 aa, Barley
LLGGVQDAPAGRENDLETIELARFAVAEHNAKANALLEFEKLVKVRQQVVAGCMHYFTIEVKEGGAKKLYEAKVWEKAWENFKQLQEFKP

SEQ ID NO: 18 - DNA, 1030 bp, Colocasia esculenta cv. Kaosiung no. 1
ctctctatagggtttgagcggccgcccgggcaggtcgcagcatcagtcgcggtgcttctgctgttgctgccaccgacctcgtcggagttcgtctcgggcgaagctttgaaggtggagtggggaggtggagacgatccagtgatcaggatggccttgatggggggcatcgtggatgtggagggagcgcagaacagcgccgaagtagaggagctcgcccgcttcgccgtcgacgaacacaataagaaggagaatgcgcttctgcagttctcccggcttgtgaaggcgaagcaacaggtggtttcgggcattatgcatcacctcactgtggaggttatcgaaggtggcaagaagaaagtgtatgaagccaaggtctgggtccaggcttggcttaactccaagaagcttcatgagttcagccccattggcgattcatcctcggttacgccagcagatctcggtgtaaaacgggatgcgcacgaagcggaatggctagagataccaacacatgatcctgtcgtccaagacgcagcaaaccatgcggtgaagagcatccagcaaagatccaatactttgttcccttatgaactgttggagatccttcatgcaaaggctaaggtgcttgaggacctcgccaaaatccatttgctactcaaacttaagaggggaagcagggaggagaagttcaaggtggaagtgcacaagaacattgaggggactttccatctgaatcagatggagcaagatcattcagactctggaaactaggatcgacggccgggtaagtgcttcgtgcacctgtaaaaaaaataattcgagtgtctgtattcccttatcatcgtactagcgtgtgtgaaaatgacaaaaacagtgctgtggagctttgcttatgatcctttattcgcctcgtactctttgactatgtctgcatcttttcctcctgcgatatttgcatgtatctcttcccgacattaagatttctatatacgaatatttggtaatcggccaaaaaaaaaaaaaaaaaaaaaaaaaa

(19) United States
(12) Patent Application Publication
(10) Pub. No.: US 2007/0162998 A1
(43) Pub. Date: Jul. 12, 2007

(54) CeCPI: taro cysteine protease inhibitor

(75) Inventors: Kai-Wun Yeh, Taipei (TW); Ai-Hua Yang, Taipei (TW)

Correspondence Address: BIRCH STEWART KOLASCH & BIRCH, PO BOX 747, FALLS CHURCH, VA, 22040-0747, US

(73) Assignee: Kai-Wun YEH

(21) Appl. No.: 11/328,078

(22) Filed: Jan. 10, 2006

(51) Int. Cl. A01H 1/00; C12Q 1/68; C07H 21/04; C12N 9/99; C12N 5/04; C12N 15/82

(52) U.S. Cl. 800/279; 435/6; 435/69.2; 435/184; 435/419; 435/468; 536/23.2

(57) ABSTRACT

An isolated polypeptide, comprising an amino acid sequence that is either the amino acid Sequence of SEQ ID NO: 2, or the amino acid sequence of amino acid residues 49 to 53 of SEQ ID NO: 2.

### BACKGROUND OF THE INVENTION

1. Field of the Invention

The present invention relates to a novel polypeptide, designated “CeCPI,” and more particularly to CeCPI fusion protein, nucleic acid molecules encoding such polypeptides and proteins, methods of using these amino acid and nucleotide sequences, and composition including these amino acid sequences.

2. Description of the Prior Art

Cystatins are proteinaceous inhibitors of cystein proteases identified in animals, as well as in monocotyledoneous and dicotyledoneous plants. Moreover, plants cystein protease inhibitors are thought to play an important role in defense mechanisms against insect and pathogen attack. In fact, several cystatins can inhibit in vitro digestive proteases from coleopteran insects (Zhao et al., Plant Physiol 111: 1299-1306 (1996)), and transgenic plants overexpressing cystatins and showing enhanced resistance against insects and nematodes have been reported, for example, by Urwin et al., Plant J. 12:455-461 (1997) which is incorporated herein by reference. These proteins are ubiquitous in the plant kingdom and have attracted the attention of researchers due to their capacity to inhibit proteases that occur not only in many herbivorous insect species but also in pathogenic fungi (Ryan, Annu Rev Phytopathol 28:425-449 (1990)). In general, these proteins are specially present in storage organs, and their synthesis might be induced systemically or locally by cell damage that contributes to the complex defense mechanisms of plants.

Cystatins inhibit sulfhydryl proteinase activities and have mainly been studied in animal cells. Similarities in their primary structures and functions show that cystatins form a single evolutionary superfamily (see, for example, Barrett, Trends Biochem Sci 12:193-196 (1987), incorporated herein by reference) that comprises three families: family-I cystatins (stefins) are about 100 aa long with no disulfide bonds; family-II cystatins (cystatin II) are about 150 aa long with two disulfide bonds in the carboxy-terminal region of the protein; and family-III cystatins (the kininogens) three regions with two disulfide loops, similar to the carboxy terminal domain found in members of the cystatin family.

In the plant kingdom, a large number of cysteine proteinase inhibitors have been discovered and these proteinase inhibitors of plant origin have been grouped into a fourth cystatin family, the “phytocystatin,” based on sequence similarities and the absence of disulfide bonds (see, Abe et al., J Biol Chem 262:16793-16797 (1987); Abe et al., Eur J Biochem 209:933-937 (1992)). Phytocystatins are single polypeptide chains with molecular masses from 12 kDa to 16 kDa and share three conserved sequence motifs. Three important regions of the mature cystatin are: a conserved Gly in the vicinity of the N terminal region, a highly conserved Gln-Xaa-Val-Xaa-Gly motif in a central loop segment, and a Pro-Trp residue in what could be the second hairpin loop. In addition, phytocystatins possess a conserved LARFAVDEHN sequence in the N-terminal region that is absent in animal cystatins.

Several phytocystatin members have been isolated from many species such as rice seeds, soybean, maize, tomato, potato, Chinese cabbage, and chestnut. Phytocystatins show variable expression patterns during plant development and defense responses to biotic and abiotic stresses (see, Felton G W and Korth K L, Curr Opin Plant Biol 3:309-314 (2000)). The physiological function of these proteins is not well understood. However, at least two functions have been proposed: regulation of protein turnover and protecting plants against insects and pathogens (see, Turk V, and Bode W, FEBS Lett 285:213-219 (1991)).

The ingestion of protease inhibitors interferes with the protein degradation process in the insect's midgut. Cystatins have been shown to inhibit the activity of digestive proteases from coleopteran pests in vitro, as well as larval development in vivo. Thus, cystatins function as “toxins” by targeting the major proteolytic digestive enzymes of herbivore insects (see Hines et al., J Agric Food Chem 39:1515-1520 (1991); Leplé et al., Mol Breed 1:319-328 (1995); Zhao et al., Plant Physiol 111:1299-1306 (1996)). Moreover, cysteine proteases play an important role in virus replication, and this has been proved in induced virus resistance in tobacco by the expression of rice cystatin (see, Gutierrez-Campos et al., Nat Biotechnol 17:1223-1226 (1999)).

The taro, Colocasia esculenta (Kaoshiung no. 1), is an important staple food of Taiwan aborigines, and is widely cultivated in local mountainous farms. This crop is popular for its high productivity and less pathogen attacks. The reason behind our investigation was the resistant mechanism in taro. In a preliminary survey on proteinase inhibitors from taro, a cysteine proteinase inhibitor with a copious amount in tuber organ was discovered.

Proteins capable of inhibiting the growth of fungus are thought to be useful in agriculture and human life.

### SUMMARY OF THE INVENTION

Accordingly, the present invention provides a novel polypeptide which has antifungal activity, designated “CeCPI.” The present invention also provides CeCPI polypeptides and CeCPI fusion proteins, nucleic acid molecules encoding such polypeptides and proteins. Moreover, the present invention provides methods of obtaining these amino acid and nucleotide sequences.

### BRIEF DESCRIPTION OF THE APPENDED DRAWINGS

The invention now being generally described, the same will be better understood by reference to the following detailed description of specific embodiments in combination with the figures that form part of this specification, wherein:

FIG. 1 depicts the nucleotide sequence of CeCPI cDNA, and its deduced amino acid sequence (GenBank accession number AF525880). The nucleotide sequence is numbered on the left and the deduced amino acid sequence is numbered on the right. The termination codon is indicated by an asterisk. Four highly conserved cystatin signatures are boxed. A potential polyadenylation signal sequence is underlined.

FIG. 2 illustrates alignment of the amino acid sequence of CeCPI with soybean, Brassica, Arabidopsis, castor bean, OCI (oryzacystatin I), OCII (oryzacystatin II), maize I, maize II and barley. Identical amino acid residues are marked with a black background.

FIG. 3 is a schematic drawing depicting analysis of the protein expression, purification, and Western blot of the GST-CeCPI fusion proteins from E. coli. Overexpressed recombinant GST-CeCPI proteins were harvested, analyzed on 15% SDS-PAGE, transferred onto a PVDF membrane, and immunostained with anti-CeCPI antiserum. (A) Coomassie blue staining of SDS-PAGE (15%). Lane M Protein marker (Bio-Rad), lane a crude extracts of uninduced bacterial culture, lane b crude extracts of bacterial culture induced by 0.1 mM IPTG, lane c puri.ed GST-CeCPI fusion protein, lane d free CeCPI protein and GST protein cleaved from GST-fusion protein by thrombin, lane e GST protein only. The overexpressed GST-CeCPI fusion protein is indicated by an arrowhead. (B) Western blot analysis. Protein samples were analyzed on SDS-PAGE, then they were blotted and immunoreacted with anti-CeCPI antiserum. Lane a′,b′,c′,d′ and e′ are the samples corresponding to those shown in (A).

FIG. 4 depicts the assays of inhibitory activity and heat stability of recombinant CeCPI. (A) Purified recombinant CeCPI proteins of different concentrations (10-200 lg) were reacted with papain of a regular quantity (20 nmol; 7×10⁻³ units) to test CeCPI's inhibitory activities. Gel activity staining was performed as described in Materials and methods. Lane M LMW protein marker (Bio-Rad), lane P papain with 20 nmol (0.5 lg), lanes 10, 20, 30, 40, 50, 100 and 200 represent the indicated amount of recombinant CeCPI protein samples used to react against a regular quantity of papain (20 nmol). (B) The residual inhibitory activity of CeCPI after heat treatment was represented by inhibition percentage. Different amounts of CeCPI protein samples (10-500 μg of GST-CeCPI) were initially heat-treated at 25, 60 and 100° C. respectively, then reacted with papain (2.5 μg) at 37° C. for 10 min to assay its residual inhibitory activity.

FIG. 5 illustrates the growth inhibition assay of phytopathogenic fungi, S. rolfsii by recombinant GST-CeCPI. Test cultures were kept at 28° C. under continuous shaking (150 rpm) for 72 h. (A) Fungal culture growing with different dosages: 0-200 μg/ml of GST-CeCPI protein. 0 Culture without tarocystatin protein, CK culture growing with 200 μg/ml GST protein. (B) Photomicrographs of mycelial morphology; cultures growing with different concentrations of CeCPI recombinant protein. CK Culture added with 200 μg of GST protein. The images were made by photographed white light microscopy.

FIG. 6 shows the microscopic observation of mycelium morphology inhibited by recombinant GST-CeCPI protein. Three fungal pathogens, A. brassicae, Rhizoctonia solani and P. aphanidermatum, were photographed under light microscopy (×160) showing retardation of mycelium growth at a concentration of 200 μg/ml.

FIG. 7 illustrates the inhibition test of recombinant GST-CeCPI protein on fungal endogenous cysteine proteinase-like extract from S. rolfsii. Protein sample (30 μg each) extracted from mycelium of S. rofsii was reacted with various concentrations (50, 100 and 150 μg, respectively) of recombinant CeCPI, then loaded on 0.1% gelatin/SDS-PAGE to visualize the cysteine protease activity. E64 30 μg crude fungal protein sample reacted with 10 μl of 10 mM E64 (Sigma), SCL 30 μg crude fungal protein extract from S. rolfsii resolved in gel alone.

### DETAILED DESCRIPTION OF THE INVENTION

Methods used in the present invention are described by inventors Yeh and co-workers in Planta 221:493-501 (2005), the entire contents of which are hereby incorporated by reference.

In the present invention, degenerated primers (SEQ ID NO: 3, SEQ ID NO:4) were used to amplify cDNA fragments which were initially reverse-transcribed from the poly(A)⁺ RNA of taro (C. esculenta cv. Kaoshiung no. 1). The specific cDNA fragments were further extended to full-length cDNA genes by using 5′- and 3′-RACE. Based on the sequence analysis data, a cDNA clone, denoted as CeCPI (SEQ ID NO: 1), was confirmed as a phytocystatin gene. The primary structure of CeCPI exhibits all the consensus sequences conserved in all phytocystatins, such as glycine residue and a conserved LARFAVDEHN (SEQ ID NO:5) in the N-terminal region, a general active motif of QXVXG (Gln⁴⁹-Val⁵⁰-Val⁵¹-Ser⁵²-Gly⁵³) (SEQ ID NO: 6), and several specific amino acid residues of phenylalanine, tyrosine, and tryptophan in β-sheet regions. The ORF of CeCPI was subcloned into expression vector, pGEX-2TK, to produce recombinant protein (SEQ ID NO: 2). The overexpressed protein was assayed on 0.1% gelatin/SDS-PAGE, and a conspicuous concentration-dependent inhibitory activity towards papain was observed. This demonstrated that CeCPI is certainly a novel phytocystatin from taro. Most plant cystatins are reported as 12-16 kDa in size, and occur commonly in Monocots, such as wheat, rice, maize, and sugarcane. However, it is interesting to note that CeCPI (SEQ ID NO: 2), a Monocot cystatin from taro, is predicted to exceed 22 kDa. Phylogenetic analysis of sequence alignments showed that the primary structure has a closer relationship with Eudicots than with Monocots, and with a longer extension of the C-terminal amino acid cluster than Eudicots. A hypothetical interpretation for this case is natural horizontal gene transfer and this mechanism, enabling a gene transfer across groups of seed plants, has been reported in higher plants.

Sclerotium rofsii Sacc. is a severe phytopathogenic fungi in tropical regions that causes great southern blight damage to tomato, peanut and banana. Therefore, it is a prime target in the antifungal survey. The antifungal activity of tarocystatin protein showed a striking retardation on mycelium growth of S. rofsii Sacc. greater than 80 μml, preferred greater than 150 μg/ml, and most preferred greater than 200 μg/ml. In this antifungal test, recombinant fusion protein, composed of GST and CeCPI protein, was used for assay. Initially, GST was suspected of toxicity action synergistic with CeCPI protein in inhibiting hyphae growth. Eventually, it was proved ineffective, because assays employing GST protein alone did not exhibit this effect. In fact, the effective dosage of antifungal activity of tarocystatin on S. rofsii is half of the above-mentioned concentration. In the antimicrobial assays, it was shown that tarocystatin is unable to inhibit the growth of E. carotovora. This is coincident with the condition that E. carotovora is the major bacterial pathogen of taro in the farm, which causes leaf blight. On the other hand, S. Rofsii causes southern blot in the tuber; it is only at the post-harvest stage that the stored condition is in a warm and humid state. In this case, tarocystatin is gradually degraded; tuber thus loses the resistance to fungal attack. It could be the case that S. Rofsii becomes a pathogen to taro. Therefore, our conclusion is that the effectiveness of cystatin is closely correlated with pathogenic resistance.

In this invention, cysteine protease is discovered to exist in S. rofsii mycelium. Additionally, an inhibitory effect of tarocystatin on fungal cysteine protease was clearly confirmed from 0.1% gelatin/SDS-PAGE assay. It is to say that CeCPI exhibits strong antifungal activity on several ubiquitous phytopathogenic fungi, such as S. rofsii Sacc. etc. Moreover, CeCPI is able to block the endogenous cysteine protease of the fungal mycelium. These results imply that the CeCPI gene has the potential to be developed into a fungicidal compound.

### EXAMPLES

### Example 1

### Molecular Cloning and Characterization of CeCPI

The taro, cultivar C. esculenta cv. Kaoshiung no. 1, was used in this study. The plants were maintained at the experimental farm of the Kaoshiung District Agricultural Improvement Station, Taiwan. Corms ˜0.5 kg were harvested, frozen in liquid nitrogen, and stored at −75° C. until used for protein and RNA extraction.

Total RNA was extracted from mature taro corms following the method described by Yeh et al. Focus 13:102-103 (1991) which is incorporated herein by reference. Poly (A)⁺ RNA was isolated using the mRNA purification kit (Amersham Pharmacia Biotech, USA). For the molecular cloning of tarocystatin gene, a strategy was performed as follows: a 0.7 kb cDNA fragment was pre-amplified from mRNA using RT-PCR with one adaptor-primer, supplied with the commercial kit (Marathoon, Clontech, Calif., USA), and one of the two cystatin degenerated primers GSP-1 and GSP-2.

(SEQ ID NO:3)

(GSP-1: 5′-(A/G)(A/G)(C/G)CTCGC(C/T/G)CG(C/A)TTCGC
CG-3′;
and

(SEQ ID NO:4)

GSP-2: 5′-CGCGTCGA(T/C)GA(A/G)CACAAC-3′.

Degenerated primers were designed based on the conserved sequence, LARFAVDEHNKK, commonly present in most of these phytocystatins. The 0.7 kb cDNA fragment was cloned into pGEM-T easy vector, and the sequence was determined and confirmed to be a tarocystatin gene. Then, 5′-RACE/3′-RACE methods were employed to extend the fragment into a full-length cDNA gene. The obtained and characterized cDNA clone, denoted as CeCPI, was deposited in GenBank under the accession number AF525880.

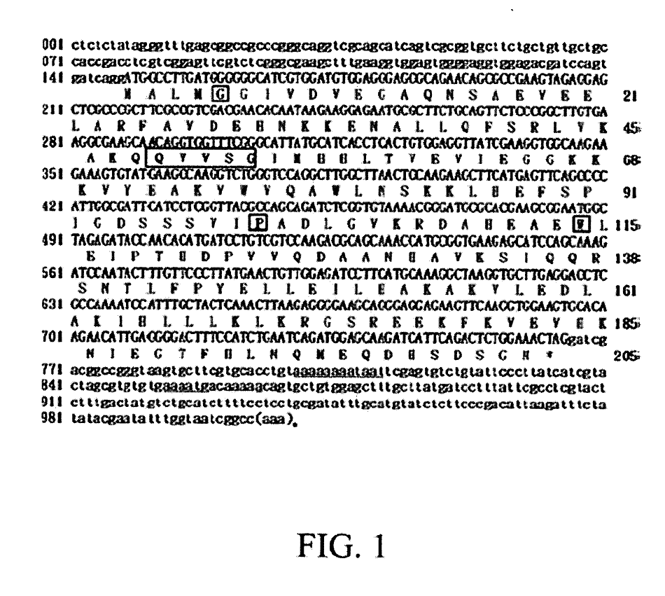

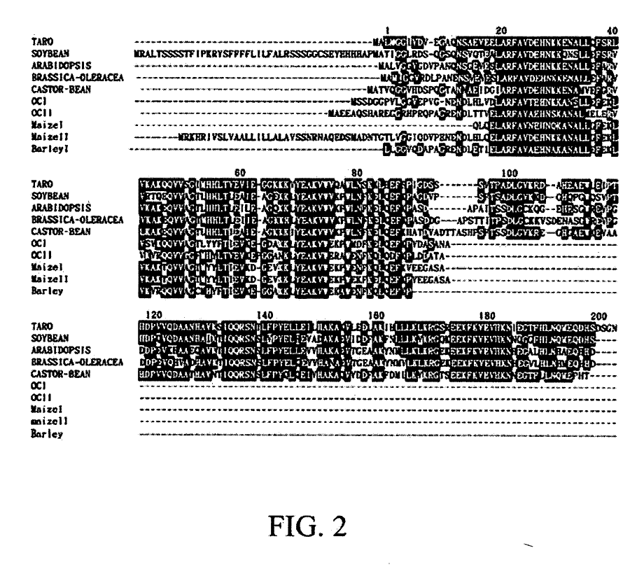

Please refer to FIG. 1. The full-length cDNA of CeCPI comprises 1,008 bp with an open reading frame of 618 nucleotides and two putative polyadenylation signals in the 3′-untranslated region. The nucleotide sequence was aligned with those of other phytocystatins in the data bank and this alignment suggested that CeCPI is a cystatin, since it shows conserved regions as expressed by other related proteins. Essential structural motifs commonly found in phytocystatin families, such as glycine (position 5) and the conserved LARFAVDEHN (position 22 to 31) (SEQ ID NO: 5) in the N-terminal region, the putative reactive domain QXVXG (Gln⁴⁹-Val⁵⁰-Val⁵¹-Ser⁵²-Gly⁵³) (SEQ ID NO: 6) in the middle region of the first hairpin loop (position 49-53), and Trp residue in the C-terminal region (position 113) are conserved in the amino acid sequence of taro CeCPI. The deduced amino acid sequence containing 205 residues shares 65.4, 64.8, 64.3, 60.5, 61.0 and 59.5% sequence identity with cystatins from soybean, Arabidopsis, field mustard, Brassica, turnip and castor bean, respectively (as shown in FIG. 2). In addition, similarities with those cystatins of monocot of OC-I, and OC-II were 50.5% (48/95) and 56.32% (49/87) respectively, as well as 60% (57/95), 57% (55/95) and 61% (55/90) with maize I, maize II and barley, respectively. It is interesting to note that sequence homology is higher with Eudicot than with Monocot, although CeCPI comes from taro (Monocot). Also, the molecular mass (MW) is more similar to that of Eudicot than to Monocot, due to a longer extension at the C-terminal end of taro CeCPI.

### Example 2

### Expression and Purification of the Recombinant CeCPI Protein

The coding region of tarocystatin gene, CeCPI, was amplified by PCR with the following primers:

The forward primer, CeCPI-F: 5′-TTGATCCATGCTTGATGGGGGG CAT-3′ (SEQ ID NO: 7); and

the reverse primer, CeCPI-R: 5′-TTGAATCCTTTCCAGAGTCTGAAT GATC-3′ (SEQ ID NO: 8).

In the amplification reaction, 20 ng template cDNA, 0.75 U Taq DNA polymerase (New England Biolabs), 1×PCR buffer, 1 mM MgCl₂, and 0.2 mM dNTPs were used. The reaction was performed in a TouchDown research thermocycler programmed for 30 cycles at 94° C. for 1 min, 46° C. for 40 s, 72° C. for 2 min, and a final extension at 72° C. for 4 min. A DNA fragment of 618 bp was purified and cleaved with the restriction enzymes BamHI and EcoRI and inserted into the expression vector pGEX-2TK (Pharmacia, USA). The recombinant clones obtained in E. coli were identified by sequence determination using an ABI Prism 377 DNA sequencer.

Furthermore, transformed E. coli cells harboring expression vector pGEX-CeCPI were cultured in LB broth containing ampicillin (100 μg/ml) and incubated at 37° C. overnight under continuous agitation. When the culture reached of OD₆₀₀=0.5-1.0, isopropyl-b-D-thiogalactocide (IPTG) was added to a final concentration at 0.1 mM to induce expression of recombinant tarocystatin protein. Four hours after IPTG induction, the cell culture was harvested and the cell pellet was suspended in 1×PBS buffer. Total soluble protein was obtained by rupturing the cell, and the soluble recombinant glutathione-S-transferase-taro cysteine protease inhibitors (GST-CeCPI) fusion protein was purified by glutathione affinity chromatography following the instructions of the B-PER GST spin purification kit (Pierce Biotechnology, USA). Furthermore, digestion by thrombin to separate CeCPI from GST was performed for 16 h. SDS-PAGE analysis of recombinant CeCPI and Western blot with anti-CeCPI antiserum were carried out as the standard molecular methods.

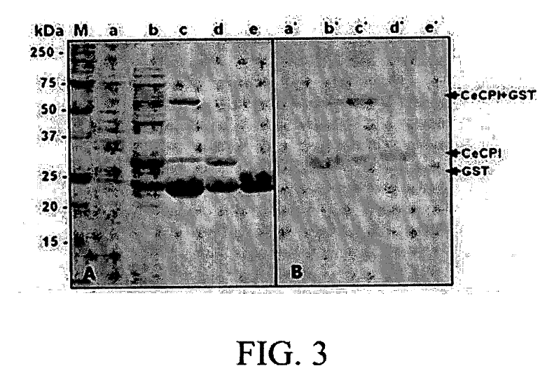

Expression plasmid pGEX-CeCPI, which harbors the open reading frame of CeCPI cDNA gene, was introduced into E. coli strain XL1-blue. The overexpression of CeCPI protein was induced by adding IPTG (0.1 mM, final conc.) to the culture medium. Total soluble proteins were harvested at 3-4 h after induction. After purification by glutathione affinity chromatography and cleavage by thrombin, the recombinant CeCPI proteins were analyzed on 15% SDS-PAGE. Electrophoresis of recombinant proteins clearly showed a highly productive expressed protein approximately 29 kDa in size (FIG. 3A). Western blotting analysis, immunostaining the CeCPI recombinant proteins with an anti-CeCPI antiserum, showed a positive signal, and further confirmed the identity (FIG. 3B).

### Example 3

### Inhibitory Activity and Heat Stability of the Recombinant Protein

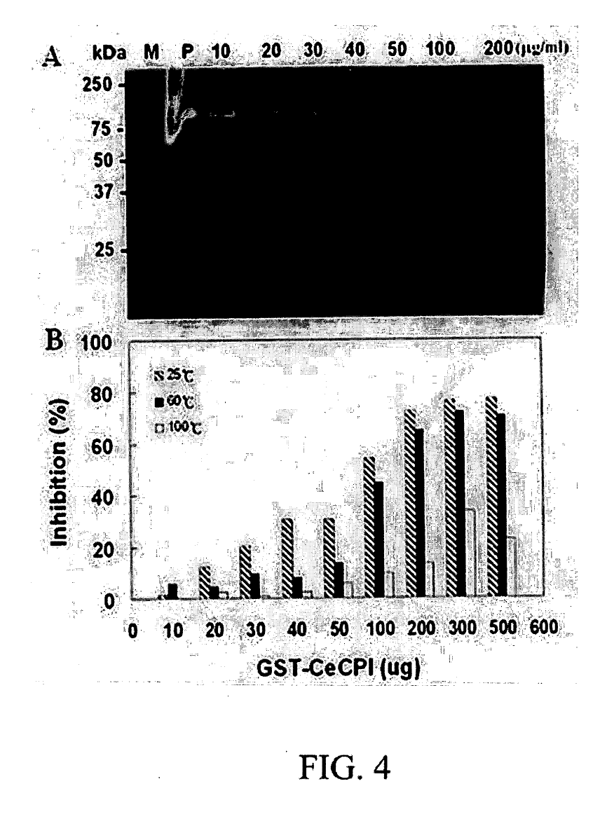

To determine whether CeCPI recombinant protein, produced from E. coli, retains an inhibitory activity against papain (cysteine protease), 0.1% gelatin/SDS polyacrylamide gel electrophoreses was employed. Different amounts of recombinant CeCPI proteins from 10 to 200 μg were pre-mixed with papain (20 nmol, 0.5 μg), incubated for 15 min at 37° C., and then resolved on 0.1% gelatin/SDS-PAGE to observe the residual protease activity of papain. The mixtures were first subjected to electrophoresis using a Hoefer SE250 system. After migration, the gels were transferred to a 2.5% v/v aqueous solution of Triton X-100 for 30 min at room temperature to allow renaturation, and incubating at active buffer (100 mM sodium phosphate pH 6.8; 8 mM EDTA; 10 mM _{L}-cysteine and 0.2% Triton X-100) for 75 min at 37° C. Subsequently, they were rinsed with water and stained with Coomassie brilliant blue. As shown in FIG. 4A, protease activities of papain appearing in 0.1% gelatin/SDS-PAGE gradually weakened as the CeCPI protein concentration was increased in the reaction and this indicates that the recombinant CeCPI protein, overexpressed in E. coli, was effective in inhibiting cysteine protease. Above all, the recombinant CeCPI at 100 μg was capable of completely depleting papain activity (20 nmol).

Various concentrations of CeCPI protein samples in 0.2 ml were mixed with 0.1 ml sodium phosphate buffer (0.5 M sodium phosphate/10 mM EDTA, pH 6.0), 0.1 ml of 2-mercaptoethanol (50 mM), and 0.1 ml papain solution (25 μg/ml), and the mixture was incubated at 37° C. for 10 min. After that, 0.2 ml of 1 mM N-benzoyl-_{DL}-arginine-2-naphthylamide (BANA) was added to start the reaction, and the mixture was incubated for another 20 min at 37° C. The reaction was terminated by adding 1 ml of 2% HCl/ethanol and 1 ml of 0.06% p-dimethylaminocinnamaldehyde/ethanol, and the mixture was allowed to stand at room temperature for 30 min for color development and finally measured at OD₅₄₀ nm. The inhibitory activity of CeCPI was recorded as an inhibition percentage (%), and the inhibition percentage (I%) of papain by CeCPI was calculated using the following equation:

$$I \% = \frac{( T - {T}^{*} )}{T} \times 100 \%$$

where T denotes the OD₅₄₀ in the absence of CeCPI and T* that in the presence of CeCPI. One inhibition unit was defined as the amount of inhibitor required to completely inhibit 2.5 μg of papain.

The heat stability of recombinant CeCPI at different temperatures was also investigated. This activity was observed from its residual inhibitory activity against papain after treating the protein samples (from 10 μg to 500 μg GST-CeCPI fusion protein) at 25, 60 and 100° C. for 5 min, respectively. This demonstrated that GST-CeCPI recombinant protein lost significant inhibitory activity only when treated at 100° C. for 5 min (FIG. 4B). Inhibition percentage (%) from the 60° C. treatment was nearly equivalent to the treatment at 25° C. However, heat treatment at 100° C. for 5 min severely decreased the GST-CeCPI inhibition percentage.

### Example 4

### Antifungal Activity and Antagonistic Mechanism

Two taro pathogens were preferentially chosen for the growth inhibition assay. One is Sclerotium rolfsii, a fungal pathogen causing stored tubers southern blot in humid and warm conditions. The other is Erwinwa carotovora subsp. carotovora, a bacterial pathogen causing soft rot of leaf and stem during farm growth. For a general survey of the antimicrobial toxicity of tarocystatin, several widespread phytopathogenic fungi were further chosen for study. They were Alternaria brassicae, Glomerella cingulata, Fusarium oxysporum, Pythium aphanidermatum and Rhizoctonia solani.AG4.

Fungal strains from the laboratory collection were grown in potato dextrose agar (PDA) medium for 7˜10 days. With the exception of S. rolfsii, which was inoculated to 2 ml of ⅓×potato dextrose broth (PDB) with five pieces of sclerotinia, a spore suspension (asexual spore) of the other fungal strains was collected for the inoculum by washing the mycelium colony with sterile ddH₂O. Approximately 10³ spores of each fungal strain were inoculated in 2 ml of ⅓×PDB, which contained various amounts of purified GST-cystatin fused proteins (with concentrations of 0, 20, 60, 80, 150, or 200 μg/ml respectively). They were incubated at 28° C. under continuous shaking (200 rpm/min) for 24˜72 h. Pathogenic bacteria (Erwinia carotovora) of taro soft rot was cultured in 1 ml of ⅓×PDB, and allowed to grow until it reached OD₆₀₀=0.2˜0.4. Then, various concentrations of fusion protein were added and incubated at 28˜30° C. for 20˜24 h. An inhibition test of tarocystatin on fungal cysteine protease activity was carried out as follows. Sclerotinia of 5-day-growing fungal cultures were harvested (0.2 g), ground in liquid nitrogen, and extracted in 500 μl of 100 mM citrate phosphate buffer at pH 6.0. After incubation on ice for 30 min, the homogenate was centrifuged at 12,000 g for 30 min at 4° C., and the supernatant was measured for protein quantification following the method of Bradford (1976). A protein sample (30 μg) of the mycelium extract was used to react with different concentrations of recombinant GST-CeCPI and E64 (Michaud et al. 1996) for 15 min at 37° C. Then, it was analyzed on 0.1% gelatin/SDS-PAGE for protease activity.

Please refer to TABLE 1. The results revealed that there was no inhibitory effect on the bacterial pathogen, E. carotovora subsp. carotovora. However, a varied inhibitory level was present among the fungal pathogens (as shown in TABLE 1). This means that there is a diversity of effective toxic dosages among fungal pathogens.

TABLE 1

| | Effective dosage | | |
|---|---|---|---|
| Pathogens | 80 μg/ml | 150 μg/ml | 200 μg/ml |
| Alternaria brassicae | + | ++ | +++ |
| Fusarium oxysporum | − | ± | + |
| Glomerella cingulata | − | ± | + |
| Pythium aphanidermatum | + | ++ | +++ |
| Rhizoctonia solani | + | ++ | +++ |
| Sclerotium rolfsii | + | ++ | +++ |
| Erwinia carotovora | − | − | − |

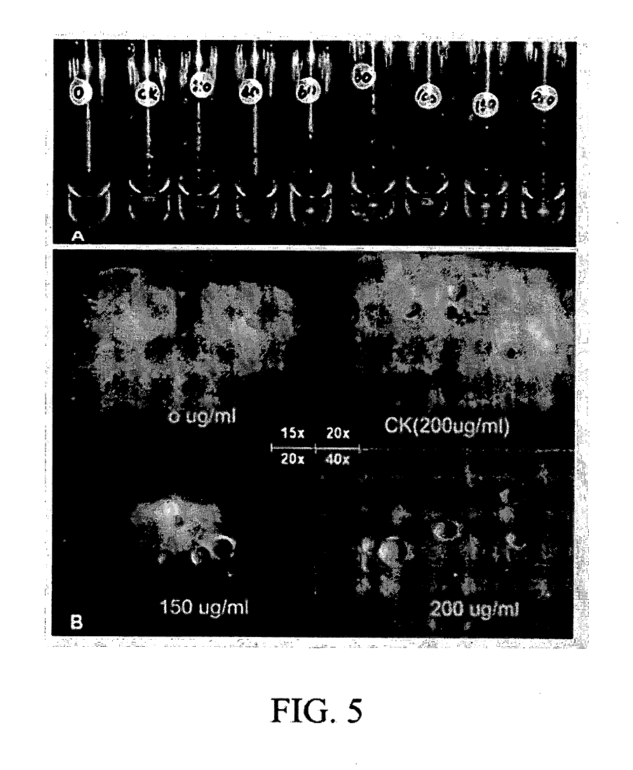

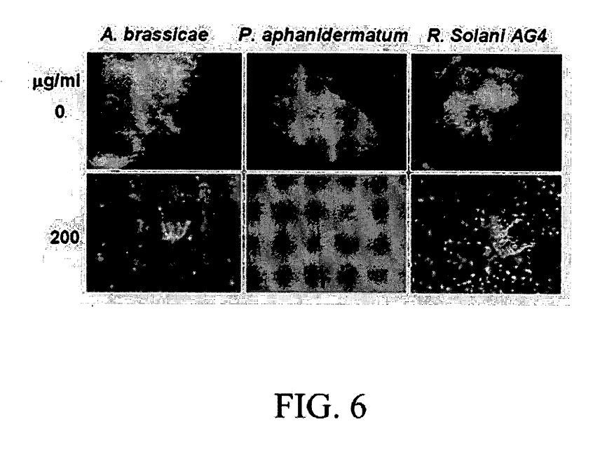

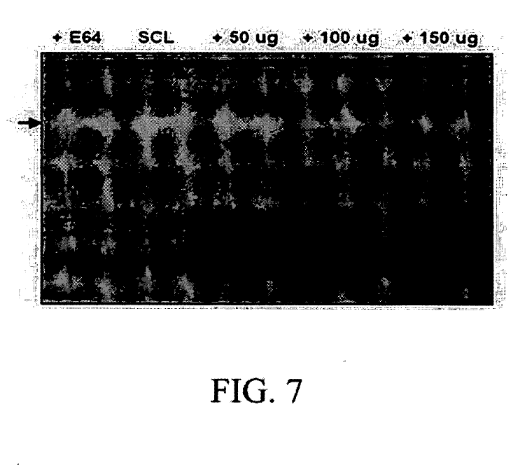

As the example of S. rolfsii, a quantitative growth inhibition of fungal mycelium was performed by incubating it with increasing amounts of purified recombinant GST-CeCPI fusion protein (20, 40, 60, 80, 100, 150 and 200 μg/ml). Abundant mycelia growth in PDB medium was found in both control cultures—without adding recombinant CeCPI or adding GST protein only (FIG. 5A). However, the mycelia growth of S. rolfsii Sacc. was strikingly inhibited at 80 μg/ml of GST-CeCPI. As the recombinant GST-CeCPI protein was applied up to 150 μg/ml or 200 μg/ml, the mycelia growth of S. rolfsii Sacc. was strongly inhibited. Hyphal morphology observed under an optical microscope (Nikon SMZ-10) showed shorter and thinner filaments (FIG. 5B). The other three fungal pathogens, i.e. A. brassicae, Rhizoctonia solani AG4 and P. aphanidermatum, showed the same inhibitory effect as exhibited by S. rolfsii (FIG. 6). To further understand the property of tarocystatin inhibiting mycelium growth, crude protein samples were extracted from the mycelia and sclerotinia of S. rofsii culture. Various concentrations (50, 100 and 150 μg) of recombinant GST-CeCPI protein and E64 (chemical inhibitor of cysteine proteinase) were reacted with 30 μg of crude protein extract of S. rofsii mycelium at 37° C. for 15 min. The mixture samples were subsequently resolved on 0.1% gelatin/SDS-PAGE to assay protease activities (FIG. 7).

The result clearly showed that crude protein sample extracted from fungal mycelium might contain cysteine proteinase. Therefore, it was able to digest gelatin contained in the running gel (FIG. 7, lane SCL). This indicates that at least one kind of cysteine protease inhibitor is indigenously present in fungal mycelium. On the other hand, the proteolytic activity of crude protein extract was accordingly blocked by the increasing amount of recombinant tarocystatin. However, E64 lacked an inhibitory effect on fungal protease activity (FIG. 7, E64). The data implied that blocking indigenous proteinase activity in fungal cells by tarocystatin is a possible mechanism for inhibiting mycelium growth. Mostly, it might come from nutrition depletion because lower protease activity in fungal cell causes less nutrition digestion and it might result in the retardation of fungal mycelium growth.

Obviously, the CeCPI of the present invention exhibits strong antifungal activity on several ubiquitous phytopathogenic fungi, such as S. rofsii Sacc. etc. These results imply that the CeCPI gene has the potential to be developed into a fungicidal agent. Furthermore, it is easy for the person skilled in the art to transform the CeCPI gene to a plant cell, so as to obtain a transgenic plant cell with antifungal activity.

It should be noted that all publications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. An isolated polypeptide, comprising an amino acid sequence that is either the amino acid sequence of SEQ ID NO: 2, or the amino acid sequence of amino acid residues 49 to 53 of SEQ ID NO: 2.

2. The isolated polypeptide of claim 1, wherein the isolated polypeptide is encoded from a nucleotide sequence comprising the nucleotide sequence of SEQ ID NO: 1.

3. The isolated polypeptide of claim 1, wherein the isolated polypeptide is encoded from a nucleotide sequence comprising nucleotides 291 to 304 of SEQ ID NO:1.

4. An isolated nucleic acid molecule, comprising a nucleotide sequence that encodes either the amino acid sequence of SEQ ID NO: 2, or the amino acid sequence of amino acid residues 49 to 53 of SEQ ID NO: 2.

5. The isolated nucleic acid molecule of claim 4, wherein the nucleic acid molecule has a nucleotide sequence comprising the nucleotide sequence of SEQ ID NO: 1.

6. The isolated nucleic acid molecule of claim 4, wherein the nucleic acid molecule comprises a nucleotide sequence comprising nucleotides 291 to 304 of SEQ ID NO:1.

7. An expression vector, comprising a nucleic acid molecule that encodes the amino acid sequence of SEQ ID NO: 2, a transcription promoter, and a transcription terminator, wherein the promoter is operably linked with the nucleic acid molecule, and wherein the nucleic acid molecule is operably linked with the transcription terminator.

8. A recombinant host cell, transformed with an expression vector, the expression vector comprising a nucleic acid molecule that encodes the amino acid sequence of SEQ ID NO: 2, a transcription promoter, and a transcription terminator, wherein the host cell is selected from the group consisting of bacterium, yeast cell, fungal cell, insect cell, avian cell, mammalian cell, and plant cell.

9. A method for producing a polypeptide comprising the amino acid sequence of SEQ ID NO: 2, comprising the steps of:
(a) extracting the nucleic acid molecule that encodes the amino acid sequence of SEQ ID NO: 2, from an organism, wherein the organism is selected from the group consisting of bacterium, animal, and plant;
(b) culturing a host cell under conditions suitable for the expression of the polypeptide; and
(c) recovering the polypeptide from the host cell culture;
wherein the host cell being transformed with an expression vector comprising a nucleic acid molecule that encodes the amino acid sequence of SEQ ID NO: 2, a transcription promoter, and a transcription terminator

10. The method of claim 9, wherein the polypeptide is encoded from a nucleotide sequence comprising nucleotides 291 to 304 of SEQ ID NO:1.

11. The method of claim 9, wherein the organism is plant.

12. The method of claim 11, wherein the plant is taro.

13. The method of claim 9, wherein the host cell is bacterium.

14. The method of claim 13, wherein the bacterium is Escherichia coli.

15. A composition, comprising a carrier, the carrier comprising a polypeptide for inhibiting the growth of fungi, wherein the polypeptide comprising an amino acid sequence that is either the amino acid sequence of SEQ ID NO: 2, or the amino acid sequence of amino acid residues 49 to 53 of SEQ ID NO: 2.

16. The composition of claim 15, wherein the polypeptide is encoded from a nucleotide sequence comprising nucleotides 291 to 304 of SEQ ID NO:1.

17. The composition of claim 15, wherein the fungi is selected from the group consisting of Alternaria brassicae, Pythium aphanidermatum, Rhizoctonia solani, and Sclerotium rolfsii.

18. The composition of claim 17, wherein a dosage of the polypeptide for inhibiting the growth of fungi is greater than 80 μg/ml.

19. The composition of claim 18, wherein the dosage of the polypeptide for inhibiting the growth of fungi is greater than 150 μg/ml.

20. A transgenic plant cell comprising a nucleotide sequence encoding a cystatin, wherein the cystatin has the amino acid sequence of SEQ ID NO: 2.
